(19) United States
(12) Patent Application Publication
(10) Pub. No.: US 2014/0031325 A1
(43) Pub. Date: Jan. 30, 2014

(54) COMBINATION THERAPY WITH LENALIDOMIDE AND A CDK INHIBITOR FOR TREATING MULTIPLE MYELOMA

(71) Applicants: Blake Bartlett, Warren, NJ (US); Maurizio Di Liberto, Rivervale, NJ (US); Xiangao Huang, Rego Park, NY (US); Selina Chen-Kiang, New York, NY (US)

(72) Inventors: Blake Bartlett, Warren, NJ (US); Maurizio Di Liberto, Rivervale, NJ (US); Xiangao Huang, Rego Park, NY (US); Selina Chen-Kiang, New York, NY (US)

(73) Assignee: Celgene Corporation, Summit, NJ (US)

(21) Appl. No.: 13/992,121

(22) PCT Filed: Dec. 5, 2011
(86) PCT No.: PCT/US11/63245

§ 371 Date: Oct. 17, 2013

Related U.S. Application Data
(60) Provisional application No. 61/420,187, filed on Dec. 6, 2010.

(51) Int. Cl. A61K 31/573; A61K 31/4439

(52) U.S. Cl. CPC A61K 31/573; A61K 31/4439
USPC 514/171

(57) ABSTRACT

Provided herein are methods for the treatment of multiple myeloma, wherein the methods comprise administration of lenalidomide and administration of a second active agent such as a CDK inhibitor.

### CROSS-REFERENCE TO RELATED APPLICATIONS

This application claims the benefit of U.S. provisional application No. 61/420,187, filed Dec. 6, 2010, the entirety of which is incorporated herein by reference.

### STATEMENT REGARDING FEDERAL FUNDING

This invention was made with government support under RO1CA120531 awarded by the National Institutes of Health. The government has certain rights in the invention.

### INTRODUCTION

Provided herein are methods for the treatment of multiple myeloma (e.g., relapsed multiple myeloma, refractory multiple myeloma, and newly diagnosed multiple myeloma), wherein the methods comprise administration of an immunomodulatory compound and administration of a cyclin-dependent kinase (CDK) inhibitor.

### BACKGROUND OF THE INVENTION

There is an enormous variety of cancers which are described in detail in the medical literature. Examples includes cancer of the lung, colon, rectum, prostate, breast, brain, and intestine. The incidence of cancer continues to climb as the general population ages, as new cancers develop, and as susceptible populations (e.g., people infected with AIDS or excessively exposed to sunlight) grow. A tremendous demand therefore exists for new methods and compositions that can be used to treat patients with cancer.

Cancer is characterized primarily by an increase in the number of abnormal cells derived from a given normal tissue, invasion of adjacent tissues by these abnormal cells, or lymphatic or blood-borne spread of malignant cells to regional lymph nodes and to distant sites (metastasis). Clinical data and molecular biologic studies indicate that cancer is a multistep process that begins with minor preneoplastic changes, which may under certain conditions progress to neoplasia. The neoplastic lesion may evolve clonally and develop an increasing capacity for invasion, growth, metastasis, and heterogeneity, especially under conditions in which the neoplastic cells escape the host's immune surveillance. Roitt, I., Brostoff, J and Kale, D., Immunology, 17.1-17.12 (3rd ed., Mosby, St. Louis, Mo., 1993).

Multiple myeloma is a hematologic cancer of plasma cells that are found in the bone marrow and produce antibodies. Kyle R. Best Pract. Res. Clin. Haematol. 2007; 20:637-664. The hallmark of myeloma is an increased production and secretion of monoclonal (M) protein into the serum and/or urine. Id. Symptoms of multiple myeloma include bone pain, infection, renal failure, anemia, and bone lesions. Types of multiple myeloma include relapsed multiple myeloma, refractory multiple myeloma, and newly diagnosed multiple myeloma. Multiple myeloma accounts for approximately 1% of all cancers but 10% of all hematologic malignancies. Rodriguez-Abreu D. Ann. Oncol. 2007; 18 (Supplement 1): i3-i8. Median age at diagnosis is 65-70 years of age. Kyle R. Best Pract Res Clin Haematol. 2007; 20:637-664.

Despite availability of a variety of chemotherapeutic agents, chemotherapy has many drawbacks. Stockdale, Medicine, vol. 3, Rubenstein and Federman, eds., ch. 12, sect. 10, 1998. Almost all chemotherapeutic agents are toxic, and chemotherapy causes significant, and often dangerous side effects including severe nausea, bone marrow depression, and immunosuppression. Additionally, even with administration of combinations of chemotherapeutic agents, many tumor cells are resistant or develop resistance to the chemotherapeutic agents. In fact, those cells resistant to the particular chemotherapeutic agents used in the treatment protocol often prove to be resistant to other drugs, even if those agents act by different mechanism from those of the drugs used in the specific treatment. This phenomenon is referred to as pleiotropic drug or multidrug resistance. Because of the drug resistance, many cancers prove refractory to standard chemotherapeutic treatment protocols.

Still, there is a significant need for safe and effective methods of treating, preventing and managing cancer, particularly multiple myeloma that are refractory to standard treatments, such as surgery, radiation therapy, chemotherapy and hormonal therapy, while reducing or avoiding the toxicities and/or side effects associated with the conventional therapies.

### SUMMARY

In certain embodiments, the present invention provides a method for treating and preventing multiple myeloma (e.g., relapsed multiple myeloma, refractory multiple myeloma, and newly diagnosed multiple myeloma), wherein the method comprises administering an immunomodulatory compound and a cyclin-dependent kinase (CDK) inhibitor. The immunomodulatory compound can be 3-(4-amino-1-oxo-1,3-dihydro-isoindol-2-yl)-piperidine-2,6-dione, also known as lenalidomide or Revlimid®. The immunomodulatory compound can be administered orally. More specifically, 3-(4-amino-1-oxo-1,3-dihydro-isoindol-2-yl)-piperidine-2,6-dione can be administered in the form of a capsule or tablet. 3-(4-Amino-1-oxo-1,3-dihydro-isoindol-2-yl)-piperidine-2,6-dione can be administered in an amount of from about 10 to about 25 mg per day. In a specific embodiment, the CDK inhibitor is highly specific inhibitor of cyclin-dependent kinase 4 (CDK4) and cyclin-dependent kinase 6 (CDK6) with potential antineoplastic activity. The CDK inhibitor can be administered orally.

In certain embodiments, additional active agents can be administered simultaneously or separately. Such additional agents include, but are not limited to, cyclophosphamide, bisphosphonate, thalidomide, dexamethasone, melphalan, prednisone, bortezomib, doxorubicin, vincristine, Vidaza® and Dacogen®.

In certain embodiments, the present invention provides a method for improving the safety and efficacy of a treatment with 3-(4-amino-1-oxo-1,3-dihydro-isoindol-2-yl)-piperidine-2,6-dione, wherein the method comprises administering a CDK inhibitor to the patient being treated with 3-(4-amino-1-oxo-1,3-dihydro-isoindol-2-yl)-piperidine-2,6-dione.

### DETAILED DESCRIPTION

6.1 Overview

Provided herein are methods for the treatment of multiple myeloma (e.g., relapsed multiple myeloma, refractory multiple myeloma, and newly diagnosed multiple myeloma), wherein the methods comprise administrations of 3-(4-amino-1-oxo-1,3-dihydro-isoindol-2-yl)-piperidine-2,6-dione and a CDK inhibitor. Certain embodiments provide administration of additional active agents, such as thalidomide, dexamethasone, melphalan, prednisone, bortezomib, cyclophosphamide, bisphosphonate, doxorubicin, vincristine, Vidaza® and Dacogen® to a subject in need of the treatment. The CDK inhibitor can be administered separately, at the same time as, or in the same pharmaceutical formulation as lenalidomide or the additional active agents. Immunomodulatory compounds are described in Section 6.2. Second active agents are described in Section 6.3. Doses and administration regimens are described in Section 6.4. Pharmaceutical compositions are described in Section 6.5.

6.2 Immunomodulatory Compounds

Immunomodulatory compounds known as IMiDs® compounds (Celgene Corporation) or Immunomodulatory Drugs can be used with second active agents in the methods and compositions of the present invention. Immunomodulatory compounds show not only potent inhibition of TNF-α but also marked inhibition of LPS induced monocyte IL1β and IL12 production. LPS induced IL6 is also inhibited by immunomodulatory compounds, albeit partially. These compounds are potent stimulators of LPS induced IL10. Id. Particular examples of IMiDs® compounds include, but are not limited to, the substituted 2-(2,6-dioxopiperidin-3-yl)-phthalimides and substituted 2-(2,6-dioxopiperidin-3-yl)-1-oxoisoindoles described in U.S. Pat. Nos. 6,281,230 and 6,316,471, both to G. W. Muller, et al.

In a specific embodiment, the IMiD® compounds that can be used with the methods and compositions of the present invention is lenalidomide (Revlimid®; 3-(4-amino-1-oxo-1,3-dihydro-isoindol-2-yl)-piperidine-2,6-dione).

Various immunomodulatory compounds contain one or more chiral centers, and can exist as racemic mixtures of enantiomers or mixtures of diastereomers. Stereomerically pure forms of such compounds, as well as the use of mixtures of those forms, can be used with the methods and compositions of the present invention. For example, mixtures comprising equal or unequal amounts of the enantiomers of a particular immunomodulatory compounds may be used in methods and compositions of the invention. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. As used herein and unless otherwise indicated, the term “stereomerically enriched” means a composition that comprises greater than about 60% by weight of one stereoisomer of a compound, preferably greater than about 70% by weight, more preferably greater than about 80% by weight of one stereoisomer of a compound. As used herein and unless otherwise indicated, the term “enantiomerically pure” means a stereomerically pure composition of a compound having one chiral center. Similarly, the term “stereomerically enriched” means a stereomerically enriched composition of a compound having one chiral center.

Compounds used with the present invention include immunomodulatory compounds that are racemic, stereomerically enriched or stereomerically pure, and pharmaceutically acceptable salts, solvates, hydrates, stereoisomers, clathrates, and prodrugs thereof. Preferred compounds used with the methods and compositions of the invention are small organic molecules having a molecular weight less than about 1,000 g/mol, and are not proteins, peptides, oligonucleotides, oligosaccharides or other macromolecules. As used herein and unless otherwise indicated, the terms “immunomodulatory compounds” and “IMiDs®” compounds (Celgene Corporation) encompasses small organic molecules that markedly inhibit TNF-α, LPS induced monocyte IL1β and IL12, and partially inhibit IL6 production. Specific immunomodulatory compounds are discussed below. TNF-α is an inflammatory cytokine produced by macrophages and monocytes during acute inflammation. TNF-α is responsible for a diverse range of signaling events within cells. TNF-α may play a pathological role in cancer. Without being limited by theory, one of the biological effects exerted by the immunomodulatory compounds is the reduction of synthesis of TNF-α. Without being bound by theory, immunomodulatory compounds enhance the degradation of TNF-α mRNA.

Further, without being limited by theory, immunomodulatory compounds used in the invention may also be potent co-stimulators of T cells and increase cell proliferation dramatically in a dose dependent manner. Immunomodulatory compounds may also have a greater co-stimulatory effect on the CD8+ T cell subset than on the CD4+ T cell subset. In addition, the compounds preferably have anti-inflammatory properties, and efficiently co-stimulate T cells.

Specific examples of immunomodulatory compounds, include, but are not limited to, cyano and carboxy derivatives of substituted styrenes such as those disclosed in U.S. Pat. No. 5,929,117; 1-oxo-2-(2,6-dioxo-3-fluoropiperidin-3yl) isoindolines and 1,3-dioxo-2-(2,6-dioxo-3-fluoropiperidine-3-yl) isoindolines such as those described in U.S. Pat. No. 5,874,448; the tetra substituted 2-(2,6-dioxopiperidin-3-yl)-1-oxoisoindolines described in U.S. Pat. No. 5,798,368; 1-oxo and 1,3-dioxo-2-(2,6-dioxopiperidin-3-yl) isoindolines (e.g., 4-methyl derivatives of thalidomide and EM-12), including, but not limited to, those disclosed in U.S. Pat. No. 5,635,517; and a class of non-polypeptide cyclic amides disclosed in U.S. Pat. Nos. 5,698,579 and 5,877,200; analogs and derivatives of thalidomide, including hydrolysis products, metabolites, derivatives and precursors of thalidomide, such as those described in U.S. Pat. Nos. 5,593,990, 5,629,327, and 6,071,948 to D'Amato; aminothalidomide, as well as analogs, hydrolysis products, metabolites, derivatives and precursors of aminothalidomide, and substituted 2-(2,6-dioxopiperidin-3-yl) phthalimides and substituted 2-(2,6-dioxopiperidin-3-yl)-1-oxoisoindoles such as those described in U.S. Pat. Nos. 6,281,230 and 6,316,471; isoindole-imide compounds such as those described in U.S. Pat. No. 7,091,353, and International Application No. PCT/US01/50401 (International Publication No. WO 02/059106). The entireties of each of the patents and patent applications identified herein are incorporated herein by reference.

Other specific immunomodulatory compounds include, but are not limited to, 1-oxo- and 1,3 dioxo-2-(2,6-dioxopiperidin-3-yl) isoindolines substituted with amino in the benzo ring as described in U.S. Pat. No. 5,635,517 which is incorporated herein by reference. These compounds have the structure I:

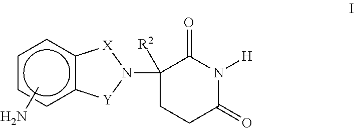

in which one of X and Y is C═O, the other of X and Y is C═O or CH₂, and R² is hydrogen or lower alkyl, in particular methyl. Specific immunomodulatory compounds include, but are not limited to:
1-oxo-2-(2,6-dioxopiperidin-3-yl)-4-aminoisoindoline;
1-oxo-2-(2,6-dioxopiperidin-3-yl)-5-aminoisoindoline;
1-oxo-2-(2,6-dioxopiperidin-3-yl)-6-aminoisoindoline;
1-oxo-2-(2,6-dioxopiperidin-3-yl)-7-aminoisoindoline;
1,3-dioxo-2-(2,6-dioxopiperidin-3-yl)-4-aminoisoindoline; and
1,3-dioxo-2-(2,6-dioxopiperidin-3-yl)-5-aminoisoindoline.

Other specific immunomodulatory compounds belong to a class of substituted 2-(2,6-dioxopiperidin-3-yl) phthalimides and substituted 2-(2,6-dioxopiperidin-3-yl)-1-oxoisoindoles, such as those described in U.S. Pat. Nos. 6,281,230; 6,316,471; 6,335,349; and 6,476,052, and International Patent Application No. PCT/US97/13375 (International Publication No. WO 98/03502), each of which is incorporated herein by reference. Compounds representative of this class are of the formulas:

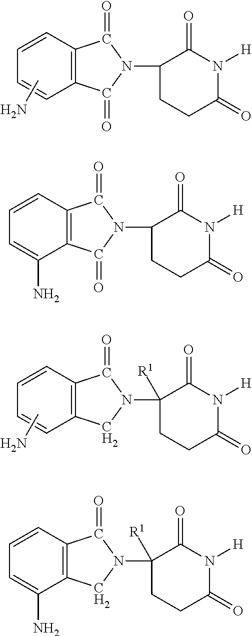

wherein R¹ is hydrogen or methyl. In a separate embodiment, the enantiomerically pure forms (e.g. optically pure (R) or (S) enantiomers) of these compounds are used with the methods and formulations of the present invention.

Still other specific immunomodulatory compounds belong to a class of isoindole-imides disclosed in U.S. patent application Ser. Nos. 10/032,286 and 09/972,487, and International Application No. PCT/US01/50401 (International Publication No. WO 02/059106), each of which are incorporated herein by reference. Representative compounds are of formula II:

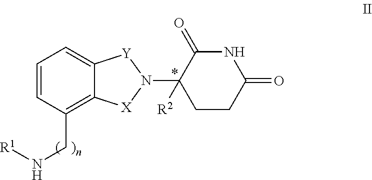

and pharmaceutically acceptable salts, hydrates, solvates, clathrates, enantiomers, diastereomers, racemates, and mixtures of stereoisomers thereof, wherein:

one of X and Y is C═O and the other is CH₂ or C═O;

R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;

R² is H, F, benzyl, (C₁-C₈)alkyl, (C₂-C₈)alkenyl, or (C₂-C₈)alkynyl;

R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;

R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;

R⁵ is (C₁-C₈)alkyl, (C₂-C₈)alkenyl, (C₂-C₈)alkynyl, benzyl, aryl, or (C₂-C₅)heteroaryl; each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group;

n is 0 or 1; and

* represents a chiral-carbon center.

In specific compounds of formula II, when n is 0 then R¹ is (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(O)OR⁴, (C₁-C₈)alkyl-N(R⁶)₂, (C₁-C₈)alkyl-OR⁵, (C₁-C₈)alkyl-C(O)OR⁵, C(S)NHR³, or (C₁-C₈)alkyl-O(CO)R⁵;

R² is H or (C₁-C₈)alkyl; and

R³ is (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₅-C₈)alkyl-N(R⁶)₂; (C₀-C₈)alkyl-NH—C(O)O—R⁵; (C₁-C₈)alkyl-OR⁵, (C₁-C₈)alkyl-C(O)OR^{S}, (C₁-C₈)alkyl-O(CO)R⁵, or C(O)OR⁵; and the other variables have the same definitions.

In other specific compounds of formula II, R² is H or (C₁-C₄)alkyl.

In other specific compounds of formula II, R¹ is (C₁-C₈)alkyl or benzyl.

In other specific compounds of formula II, R¹ is H, (C₁-C₈)alkyl, benzyl, CH₂OCH₃, CH₂CH₂OCH₃, or

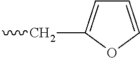

In another embodiment of the compounds of formula II, R¹ is

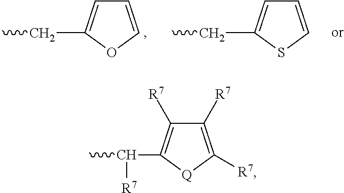

wherein Q is O or S, and each occurrence of R⁷ is independently H, (C₁-C₈)alkyl, benzyl, CH₂OCH₃, or CH₂CH₂OCH₃.

In other specific compounds of formula II, R¹ is C(O)R³.

In other specific compounds of formula II, R³ is (C₀-C₄)alkyl-(C₂-C₅)heteroaryl, (C₁-C₈)alkyl, aryl, or (C₀-C₄)alkyl-OR⁵.

In other specific compounds of formula II, heteroaryl is pyridyl, furyl, or thienyl.

In other specific compounds of formula II, R¹ is C(O)OR⁴.

In other specific compounds of formula II, the H of C(O)NHC(O) can be replaced with (C₁-C₄)alkyl, aryl, or benzyl.

Still other specific immunomodulatory compounds belong to a class of isoindole-imides disclosed in U.S. patent application Ser. No. 09/781,179, International Publication No. WO 98/54170, and U.S. Pat. No. 6,395,754, each of which are incorporated herein by reference. Representative compounds are of formula III:

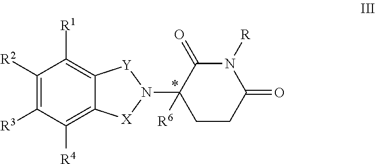

and pharmaceutically acceptable salts, hydrates, solvates, clathrates, enantiomers, diastereomers, racemates, and mixtures of stereoisomers thereof, wherein:

one of X and Y is C═O and the other is CH₂ or C═O;

R is H or CH₂OCOR′;

(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;

R⁵ is hydrogen or alkyl of 1 to 8 carbons

R⁶ hydrogen, alkyl of 1 to 8 carbon atoms, benzo, chloro, or fluoro;

R′ is R⁷—CHR¹⁰—N(R⁸R⁹);

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂—[X]X₁CH₂CH₂— in which [X]X₁ is —O—, —S—, or —NH—;

R¹⁰ is hydrogen, alkyl of to 8 carbon atoms, or phenyl; and

* represents a chiral-carbon center.

Other immunomodulatory compounds are 4-(amino)-2-(2,6-dioxo(3-piperidyl))-isoindoline-1,3-dione and 3-(4-amino-1-oxo-1,3-dihydro-isoindol-2-yl)-piperidine-2,6-dione. The compounds can be obtained via standard, synthetic methods (see e.g., U.S. Pat. No. 5,635,517, incorporated herein by reference). The compounds are available from Celgene Corporation, Summit, N.J. 4-(Amino)-2-(2,6-dioxo(3-piperidyl))-isoindoline-1,3-dione (pomalidomide) has the following chemical structure:

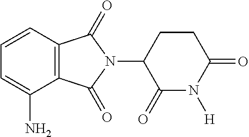 [SMILES: Nc1cccc2c1C(=O)N(C1CCC(=O)NC1=O)C2=O]

The compound 3-(4-amino-1-oxo-1,3-dihydro-isoindol-2-yl)-piperidine-2,6-dione (lenaldiomide or REVLIMID®) has the following chemical structure:

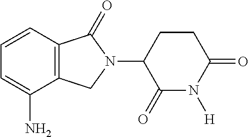 [SMILES: Nc1cccc2c1CN(C1CCC(=O)NC1=O)C2=O]

Other immunomodulatory compounds are amorphous 3-(4-amino-1-oxo-1,3 dihydro-isoindol-2-yl)-piperidine-2,6-dione, and crystalline solid forms of 3-(4-amino-1-oxo-1,3-dihydro-isoindol-2-yl)-piperidine-2,6-dione, including Form A, Form B, Form C, Form D, Form E, Form F, Form G and Form H. (see e.g., U.S. Pat. No. 7,465,800, U.S. patent, incorporated herein by reference).

In some embodiments, the immunomodulatory compound can be, for example, a compound of formula IV, V, VI, VII, VIII, IX, X, XI, XII, XIII, XIV, XV, XVI, XVII, XVIII, XIX, XX, XXI or a pharmaceutically acceptable salt, solvate, or stereoisomer thereof:

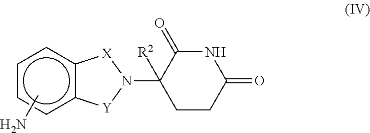

wherein:
one of X and Y is C═O, the other of X and Y is C═O or CH₂;
R² is hydrogen or lower alkyl;

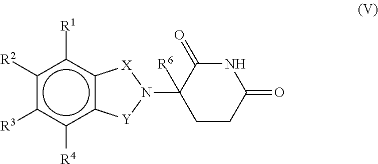

wherein:
one of X and Y is C═O and the other of X and Y is C═O or CH₂;
(i) each of R¹, R², R³, and R⁴, independently of the others, is halo, alkyl of 1 to 4 carbon atoms, or alkoxy of 1 to 4 carbon atoms, or (ii) one of R¹, R², R³, and R⁴ is —NHR⁵ and the remaining of R¹, R², R³, and R⁴ are hydrogen;
R⁵ is hydrogen or alkyl of 1 to 8 carbon atoms;
R⁶ is hydrogen, alkyl of 1 to 8 carbon atoms, benzyl, or halo;

provided that R⁶ is other than hydrogen if X and Y are C═O and (i) each of R¹, R², R³, and R⁴ is fluoro or (ii) one of R¹, R², R³, or R⁴ is amino;

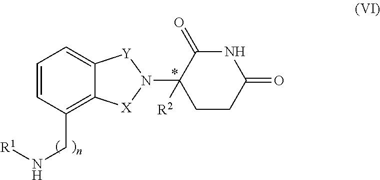

wherein:
one of X and Y is C═O and the other is CH₂ or C═O;
R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;
R² is H, F, benzyl, (C₁-C₈)alkyl, (C₂-C₈)alkenyl, or (C₂-C₈)alkynyl;
R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl—(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;
R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;
R⁵ is (C₁-C₈)alkyl, (C₂-C₈)alkenyl, (C₂-C₈)alkynyl, benzyl, aryl, or (C₂-C₅)heteroaryl;
R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group;
n is 0 or 1; and
* is a chiral-carbon center;

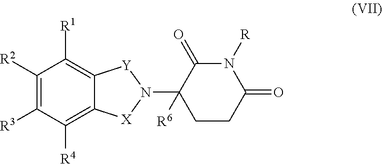

wherein:
one of X and Y is C═O and the other is CH₂ or C═O;
R is H or CH₂OCOR′;
(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;
R⁵ is hydrogen or alkyl of 1 to 8 carbons
R⁶ hydrogen, alkyl of 1 to 8 carbon atoms, benzo, chloro, or fluoro;
R′ is R⁷—CHR¹⁰—N(R⁸R⁹);
R⁷ is m-phenylene or p-phenylene or —(CₙH₂ₙ)— in which n has a value of 0 to 4;
each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X₁CH₂CH₂— in which X₁ is —O—, —S—, or —NH—;
R¹⁰ is hydrogen, alkyl of to 8 carbon atoms, or phenyl; and
* represents a chiral-carbon center;

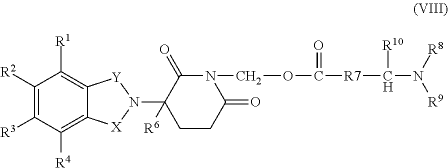

wherein:
one of X and Y is C═O and the other of X and Y is C═O or CH₂;
(i) each of R¹, R², R³, or R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;
R⁵ is hydrogen or alkyl of 1 to 8 carbon atoms;
R⁶ is hydrogen, alkyl of 1 to 8 carbon atoms, benzo, chloro, or fluoro;
R⁷ is m-phenylene or p-phenylene or —(CₙH₂ₙ)— in which n has a value of 0 to 4;
each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X′CH₂CH₂— in which X¹ is —O—, —S—, or —NH—; and

R¹⁰ is hydrogen, alkyl of to 8 carbon atoms, or phenyl;

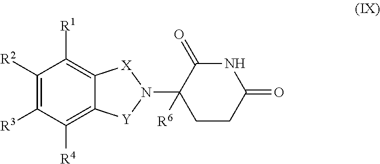

wherein:
one of X and Y is C═O and the other of X and Y is C═O or CH₂;
(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is nitro or protected amino and the remaining of R¹, R², R³, and R⁴ are hydrogen; and
R⁶ is hydrogen, alkyl of 1 to 8 carbon atoms, benzo, chloro, or fluoro;

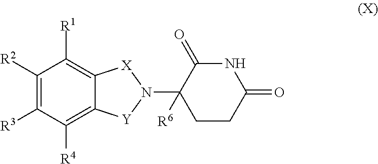

wherein:
one of X and Y is C═O and the other of X and Y is C═O or CH₂;
(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;
R⁵ is hydrogen, alkyl of 1 to 8 carbon atoms, or CO—R⁷—CH(R¹⁰)NR⁸R⁹ in which each of R⁷, R⁸, R⁹, and R¹⁰ is as herein defined; and
R⁶ is alkyl of 1 to 8 carbon atoms, benzo, chloro, or fluoro;

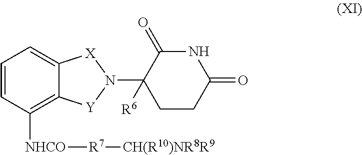

wherein:
one of X and Y is C═O and the other of X and Y is C═O or CH₂;
R⁶ is hydrogen, alkyl of 1 to 8 carbon atoms, benzyl, chloro, or fluoro;
R⁷ is m-phenylene, p-phenylene or —(CₙH₂ₙ)— in which n has a value of 0 to 4;
each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S— or —NH—; and
R¹⁰ is hydrogen, alkyl of 1 to 8 carbon atoms, or phenyl;

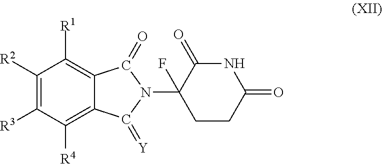

wherein:
Y is oxygen or H² and
each of R¹, R², R³, and R⁴, independently of the others, is hydrogen, halo, alkyl of 1 to 4 carbon atoms, alkoxy of 1 to 4 carbon atoms, or amino;

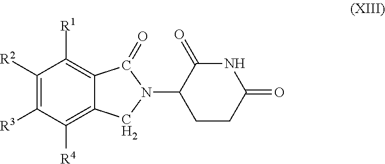

wherein:
each of R¹, R², R³, and R⁴, independently of the others, is halo, alkyl of 1 to 4 carbon atoms, or alkoxy of 1 to 4 carbon atoms;

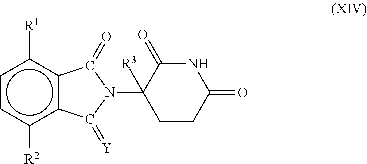

wherein:
Y is oxygen or H₂,
a first of R¹ and R² is halo, alkyl, alkoxy, alkylamino, dialkylamino, cyano, or carbamoyl, the second of R¹ and R², independently of the first, is hydrogen, halo, alkyl, alkoxy, alkylamino, dialkylamino, cyano, or carbamoyl, and
R³ is hydrogen, alkyl, or benzyl;

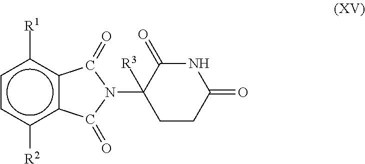

wherein:
a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl;
the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl; and
R³ is hydrogen, alkyl of from 1 to 4 carbon atoms, or benzyl;

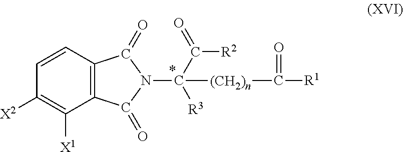

wherein:
when n is not zero and R¹ is not the same as R², C* is a center of chirality;
one of X¹ and X² is amino, nitro, alkyl of one to six carbons, or NH—Z, and the other of X¹ or X² is hydrogen;
each of R¹ and R² independent of the other, is hydroxy or NH—Z; R³ is hydrogen, alkyl of one to six carbons, halo, or haloalkyl;
Z is hydrogen, aryl, alkyl of one to six carbons, formyl, or acyl of one to six carbons; and
n has a value of 0, 1, or 2;
   provided that if X¹ is amino, and n is 1 or 2, then R¹ and R² are not both hydroxy;

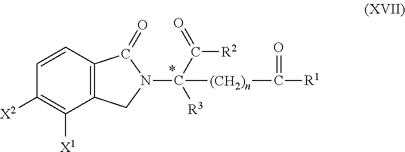

wherein:
when n is not zero and R¹ is not R², C* is a center of chirality;
one of X¹ and X² is amino, nitro, alkyl of one to six carbons, or NH—Z, and the other of X¹ or X² is hydrogen;
each of R¹ and R² independent of the other, is hydroxy or NH—Z; R³ is alkyl of one to six carbons, halo, or hydrogen;
Z is hydrogen, aryl or an alkyl or acyl of one to six carbons; and
n has a value of 0, 1, or 2;

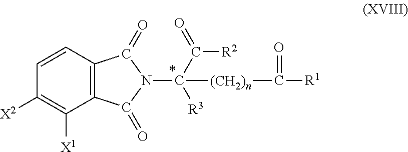

wherein:
when n is not zero and R¹ is not R², C* is a center of chirality;
one of X¹ and X² is amino, nitro, alkyl of one to six carbons, or NH—Z, and the other of X¹ or X² is hydrogen;
each of R¹ and R² independent of the other, is hydroxy or NH—Z; R³ is alkyl of one to six carbons, halo, or hydrogen;
Z is hydrogen, aryl, or an alkyl or acyl of one to six carbons; and
n has a value of 0, 1, or 2;

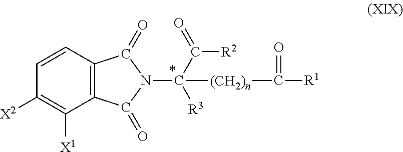

wherein:
one of X¹ and X² is nitro, or NH—Z, and the other of X¹ or X² is hydrogen;
each of R¹ and R², independent of the other, is hydroxy or NH—Z;
R³ is alkyl of one to six carbons, halo, or hydrogen;
Z is hydrogen, phenyl, an acyl of one to six carbons, or an alkyl of one to six carbons;
n has a value of 0, 1, or 2; and
if —COR² and —(CH₂)ₙCOR¹ are different, C* is a center of chirality;

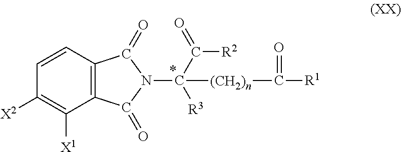

wherein:
one of X¹ and X² is alkyl of one to six carbons;
each of R¹ and R², independent of the other, is hydroxy or NH—Z;
R³ is alkyl of one to six carbons, halo, or hydrogen;
Z is hydrogen, phenyl, an acyl of one to six carbons, or an alkyl of one to six carbons;
n has a value of 0, 1, or 2; and
if —COR² and —(CH₂)ₙCOR¹ are different, C* is a center of chirality;

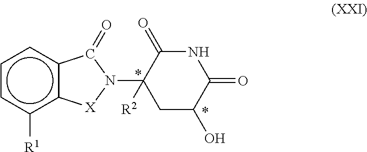

wherein:
the * carbons are centers of chirality;
X is —C(O)— or —CH₂—;
R¹ is alkyl of 1 to 8 carbon atoms or —NHR³;
R² is hydrogen, alkyl of 1 to 8 carbon atoms, or halogen; and
R³ is hydrogen, alkyl of 1 to 8 carbon atoms, unsubstituted or substituted with alkoxy of 1 to 8 carbon atoms, halo, amino, or alkylamino of 1 to 4 carbon atoms, cycloalkyl of 3 to 18 carbon atoms, phenyl, unsubstituted or substituted with alkyl of 1 to 8 carbon atoms, alkoxy of 1 to 8 carbon atoms, halo, amino, or alkylamino of 1 to 4 carbon atoms, benzyl, unsubstituted or substituted with alkyl of 1 to 8 carbon atoms, alkoxy of 1 to 8 carbon atoms, halo, amino, or alkylamino of 1 to 4 carbon atoms, or —COR⁴, wherein
R⁴ is hydrogen, alkyl of 1 to 8 carbon atoms, unsubstituted or substituted with alkoxy of 1 to 8 carbon atoms, halo, amino, or alkylamino of 1 to 4 carbon atoms, cycloalkyl of 3 to 18 carbon atoms, phenyl, unsubstituted or substituted with alkyl of 1 to 8 carbon atoms, alkoxy of 1 to 8 carbon atoms, halo, amino, or alkylamino of 1 to 4 carbon atoms, or benzyl, unsubstituted or substituted with alkyl of 1 to 8 carbon atoms, alkoxy of 1 to 8 carbon atoms, halo, amino, or alkylamino of 1 to 4 carbon atoms.

As used herein and unless otherwise indicated, the term “pharmaceutically acceptable salt” encompasses non-toxic acid and base addition salts of the compound to which the term refers. Acceptable non-toxic acid addition salts include those derived from organic and inorganic acids or bases know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

As used herein and unless otherwise indicated, the term “prodrug” means a derivative of a compound that can hydrolyze, oxidize, or otherwise react under biological conditions (in vitro or in vivo) to provide the compound. Examples of prodrugs include, but are not limited to, derivatives of immunomodulatory compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of immunomodulatory compounds that comprise —NO, —NO₂, —ONO, or —ONO₂ moieties. Prodrugs can typically be prepared using well-known methods, such as those described in 1 Burger's Medicinal Chemistry and Drug Discovery, 172-178, 949-982 (Manfred E. Wolff ed., 5th ed. 1995), and Design of Prodrugs (H. Bundgaard ed., Elselvier, N.Y. 1985).

As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxymethyl, pivaloyloxymethyl, and pivaloyloxyethyl esters), lactonyl esters (such as phthalidyl and thiophthalidyl esters), lower alkoxyacyloxyalkyl esters (such as methoxycarbonyl-oxymethyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, α-amino acid amides, alkoxyacyl amides, and alkylaminoalkylcarbonyl amides. Examples of biohydrolyzable carbamates include, but are not limited to, lower alkylamines, substituted ethylenediamines, amino acids, hydroxyalkylamines, heterocyclic and heteroaromatic amines, and polyether amines.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

6.3 Second Active Agents

Immunomodulatory compounds can be combined with other pharmacologically active compounds (“second active agents”) in methods and compositions of the invention. It is believed that certain combinations work synergistically in the treatment of particular types of cancers. Immunomodulatory compounds can also work to alleviate adverse effects associated with certain second active agents, and some second active agents can be used to alleviate adverse effects associated with immunomodulatory compounds.

One or more second active ingredients or agents can be used in the methods and compositions of the invention together with an immunomodulatory compound. Second active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies. Typical large molecule active agents are biological molecules, such as naturally occurring or artificially made proteins. Proteins that are particularly useful in this invention include proteins that stimulate the survival and/or proliferation of hematopoietic precursor cells and immunologically active poietic cells in vitro or in vivo. Others stimulate the division and differentiation of committed erythroid progenitors in cells in vitro or in vivo. Particular proteins include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alfa-n1, interferon alfa-n3, interferon beta-I a, and interferon gamma-I b; GM-CF and GM-CSF; and EPO.

Particular proteins that can be used in the methods and compositions of the invention include, but are not limited to: filgrastim, which is sold in the United States under the trade name Neupogen® (Amgen, Thousand Oaks, Calif.); sargramostim, which is sold in the United States under the trade name Leukine® (Immunex, Seattle, Wash.); and recombinant EPO, which is sold in the United States under the trade name Epogen® (Amgen, Thousand Oaks, Calif.).

Recombinant and mutated forms of GM-CSF can be prepared as described in U.S. Pat. Nos. 5,391,485; 5,393,870; and 5,229,496; all of which are incorporated herein by reference. Recombinant and mutated forms of G-CSF can be prepared as described in U.S. Pat. Nos. 4,810,643; 4,999,291; 5,528,823; and 5,580,755; all of which are incorporated herein by reference.

This invention encompasses the use of native, naturally occurring, and recombinant proteins. The invention further encompasses mutants and derivatives (e.g., modified forms) of naturally occurring proteins that exhibit, in vivo, at least some of the pharmacological activity of the proteins upon which they are based. Examples of mutants include, but are not limited to, proteins that have one or more amino acid residues that differ from the corresponding residues in the naturally occurring forms of the proteins. Also encompassed by the term “mutants” are proteins that lack carbohydrate moieties normally present in their naturally occurring forms (e.g., nonglycosylated forms). Examples of derivatives include, but are not limited to, pegylated derivatives and fusion proteins, such as proteins formed by fusing IgG1 or IgG3 to the protein or active portion of the protein of interest. See, e.g., Penichet, M. L. and Morrison, S. L., J. Immunol. Methods 248:91-101 (2001).

Antibodies that can be used in combination with compounds of the invention include monoclonal and polyclonal antibodies. Examples of antibodies include, but are not limited to, trastuzumab (Herceptin®), rituximab (Rituxan®), bevacizumab (Avastin™), pertuzumab (Omnitarg™), tositumomab (Bexxar®), edrecolomab (Panorex®), and G250. Compounds of the invention can also be combined with, or used in combination with, anti-TNF-α antibodies.

Large molecule active agents may be administered in the form of anti-cancer vaccines. For example, vaccines that secrete, or cause the secretion of, cytokines such as IL-2, G-CSF, and GM-CSF can be used in the methods, pharmaceutical compositions, and kits of the invention. See, e.g., Emens, L. A., et al., Curr. Opinion Mol. Ther. 3(1):77-84 (2001).

In one embodiment of the invention, the large molecule active agent reduces, eliminates, or prevents an adverse effect associated with the administration of an immunomodulatory compound. Depending on the particular immunomodulatory compound and the disease or disorder begin treated, adverse effects can include, but are not limited to, drowsiness and somnolence, dizziness and orthostatic hypotension, neutropenia, infections that result from neutropenia, increased HIV-viral load, bradycardia, Stevens-Johnson Syndrome and toxic epidermal necrolysis, and seizures (e.g., grand mal convulsions). A specific adverse effect is neutropenia.

Second active agents that are small molecules can also be used to alleviate adverse effects associated with the administration of an immunomodulatory compound. However, like some large molecules, many are believed to be capable of providing a synergistic effect when administered with (e.g., before, after or simultaneously) an immunomodulatory compound. Examples of small molecule second active agents include, but are not limited to, anti-cancer agents, antibiotics, immunosuppressive agents, and steroids.

In a specific embodiment, second active agents that can be used with the methods and formulations of the present invention include a CDK inhibitor, thalidomide, dexamethasone, melphalan, prednisone, bortezomib, cyclophosphamide, bisphosphonate, doxorubicin, vincristine, Vidaza® and Dacogen®.

In a specific embodiment, CDK Inhibitors can be CDK4 inhibitors, CDK6 inhibitors, or CDK4/CDK6 (CDK4/6) inhibitors. In a specific embodiment, a CDK4/CDK6 inhibitor inhibits CDK4/6-dependent pRb phosphorylation and DNA synthesis in HCT116 cells.

In a specific embodiment, a CDK4/CDK6 inhibitor is PD-0332991 having the following structure:

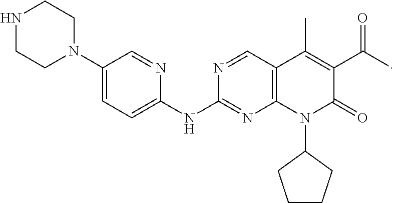

In certain embodiments, induction of prolonged early G1 arrest by a CDK4/CDK6 inhibitor markedly enhances the killing of primary bone marrow myeloma cells by proteasome inhibitors despite stromal protection. In certain embodiments, release from the G1 block upon a CDK4/CDK6 inhibitor withdraw leads to synchronous progression to S phase, which further augments cytotoxic killing of multiple myeloma cells. It is believed that acceleration of early G1 arrest by a CDK4/CDK6 inhibitor in primary bone marrow myeloma cells enhances lenalidomide killing in the presence of bone marrow stromal cells.

In a specific embodiment, Revlimid® is used with a CDK4/CDK6 inhibitor to treat multiple myeloma (e.g., relapsed multiple myeloma, refractory multiple myeloma, and newly diagnosed multiple myeloma). In a specific embodiment, Revlimid® is used with PD-0332991 to treat multiple myeloma (e.g., relapsed multiple myeloma, refractory multiple myeloma, and newly diagnosed multiple myeloma).

In a specific embodiment, one or more of the following agents: thalidomide, dexamethasone, melphalan, prednisone, bortezomib, cyclophosphamide, bisphosphonate, doxorubicin, vincristine, Vidaza® and Dacogen® are administered simulateously with or separately from a CDK4/CDK6 inhibitor and Revlimid® to treat multiple myeloma (e.g., relapsed multiple myeloma, refractory multiple myeloma, and newly diagnosed multiple myeloma).

In a specific embodiment, Revlimid® is used with low-dose dexamethasone and a CDK4/CDK6 inhibitor to treat multiple myeloma (e.g., relapsed multiple myeloma, refractory multiple myeloma, and newly diagnosed multiple myeloma). In a specific embodiment, Revlimid® is used with Velcade® and a CDK4/CDK6 inhibitor to treat multiple myeloma (e.g., relapsed multiple myeloma, refractory multiple myeloma, and newly diagnosed multiple myeloma).

In a specific embodiment, Revlimid® is used with dexamethasone, Velcade®, and a CDK4/CDK6 inhibitor to treat multiple myeloma (e.g., relapsed multiple myeloma, refractory multiple myeloma, and newly diagnosed multiple myeloma). The invention also provides a pharmaceutical composition comprising Revlimid®, dexamethasone, Velcade®, and a CDK4/CDK6 inhibitor.

In a specific embodiment, a combination of Revlimid®, melphalan, prednisone, and a CDK4/CDK6 inhibitor is used to treat multiple myeloma (e.g., relapsed multiple myeloma, refractory multiple myeloma, and newly diagnosed multiple myeloma). The invention also provides a pharmaceutical composition comprising Revlimid®, melphalan, prednisone, and a CDK4/CDK6 inhibitor.

In certain embodiments, Revlimid® and a CDK4/CDK6 inhibitor are used with doxorubicin (Doxil®), vincristine and/or dexamethasone (Decadron®) to treat multiple myeloma (e.g., relapsed multiple myeloma, refractory multiple myeloma, and newly diagnosed multiple myeloma).

6.4 Methods for Treatment and Prevention

Provided herein are methods for the treatment of multiple myeloma (e.g., relapsed multiple myeloma, refractory multiple myeloma, and newly diagnosed multiple myeloma), wherein the methods comprise administering to a patient in need of treatment an immunomodulatory compound (see Section 6.2) and administering to the patient in need of treatment a second active agent (see Section 6.3).

In certain embodiments, administration of an immunomodulatory compound and a second active agent is sufficient to ameliorate one symptom of multiple myeloma (e.g., relapsed multiple myeloma, refractory multiple myeloma, and newly diagnosed multiple myeloma). In certain embodiments, administration of an immunomodulatory compound and a second active agent is sufficient to prevent one symptom of multiple myeloma (e.g., relapsed multiple myeloma, refractory multiple myeloma, and newly diagnosed multiple myeloma) from worsening.

In certain embodiments, administration of an immunomodulatory compound and a second active agent results in at least 5%, 10%, 15%, 20%, 25%, 30%, 35%, 40%, 45%, 50%, 55%, 60%, 65%, 70%, 75%, 80%, 85%, 90%, 95%, 98%, 99%, or 100% reduction of malignant plasma cells present in the patient relative to the beginning of treatment or relative to an untreated patient.

In certain embodiments, a method of the present invention comprises administering to a patient in need of treatment a therapeutically effective amount of an immunomodulatory compound (see Section 6.2) and administering to the patient in need of treatment a therapeutically effective amount of a second active agent (see Section 6.3).

In one embodiment of the invention, an immunomodulatory compound can be administered orally and in single or divided daily doses in an amount of from about 0.10 to about 150 mg/day. In a preferred embodiment, 3-(4-amino-1-oxo-1,3-dihydro-isoindol-2-yl-piperidine-2,6-dione (lenalidomide or Revlimid®) may be administered in an amount of from about 5 to 25 mg per day for patients with multiple myeloma (e.g., relapsed multiple myeloma, refractory multiple myeloma, and newly diagnosed multiple myeloma).

In a preferred embodiment, Revlimid® may be administered in an amount of about 25 mg per day on Days 1-21 with rest on Days 22-28 in a cycle of 28 days for patients with multiple myeloma (e.g., relapsed multiple myeloma, refractory multiple myeloma, and newly diagnosed multiple myeloma).

In a particular embodiment, Revlimid® may be administered in an amount of about 25 mg per day on Days 1-21 with rest on Days 22-28 in a cycle of 28 days, and dexamethasone may be administered in an amount of about 40 mg/day on Days 1-4, 9-12, and 17-20 in a cycle of 28 days for patients with multiple myeloma (e.g., relapsed multiple myeloma, refractory multiple myeloma, and newly diagnosed multiple myeloma).

In a particular embodiment, Revlimid® may be administered in an amount of about 25 mg per day on Days 1-21 with rest on Days 22-28 in a cycle of 28 days, and dexamethasone may be administered in an amout of about 40 mg/day on Days 1, 8, 15, and 22 in a cycle of 28 days for patients with multiple myeloma (e.g., relapsed multiple myeloma, refractory multiple myeloma, and newly diagnosed multiple myeloma).

In a particular embodiment, the methods of the invention comprise administering a additional active agent, or a pharmaceutically acceptable salt, solvate, hydrate, stereoisomer, clathrate or prodrug thereof, in combination with Revlimid® and a CDK4/CDK6 inhibitor to treat or prevent multiple myeloma (e.g., relapsed multiple myeloma, refractory multiple myeloma, and newly diagnosed multiple myeloma).

Administration of a CDK4/CDK6 inhibitor and other second active agent to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the disease being treated. A preferred route of administration for an immunomodulatory compound of the invention is oral. Preferred routes of administration for the second active agents are known to those of ordinary skill in the art. See, e.g., Physicians' Desk Reference.

In one embodiment of the invention, the second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of cancers, the severity and stage of disease, and the amount(s) of immunomodulatory compounds and any optional additional active agents concurrently administered to the patient.

This invention also encompasses a method of increasing the dosage of an immunomodulatory compound that can be safely and effectively administered to a patient, by combining the administration regimen of the second active agent with an administration regimen of an immunomodulatory compound. In certain embodiments, the present invention provides a method for increasing the dose of Revlimid® that can be administered to a patient with multiple myeloma (e.g., relapsed multiple myeloma, refractory multiple myeloma, and newly diagnosed multiple myeloma) by at least 10%, 20%, 30%, 40%, 50%, 60%, 70%, 80%, 90%, 100%, 150%, 200%, 250%, 300%, 350%, 400%, 450%, 500%, 600%, 700%, 800%, 900%, or by at least 1000% without increasing the severity of any side effects or without causing any side effects.

In another embodiment, this invention encompasses a method of treating, preventing and/or managing multiple myeloma (e.g., relapsed multiple myeloma, refractory multiple myeloma, and newly diagnosed multiple myeloma) which comprises administering Revlimid® and a second active agent (e.g., CDK4/CDK6 inhibitor) in combination with conventional therapy including, but not limited to, surgery, immunotherapy, biological therapy, radiation therapy, or other non-drug based therapy presently used to treat, prevent or manage cancer. In one embodiment, an immunomodulatory compound can be administered in an amount of from about 0.1 to about 150 mg, prior to, during, or after the use of conventional therapy.

Without being limited by theory, it is believed that combination of Revlimid® and a CDK4/CDK6 inhibitor may provide additive or synergistic effects when given concurrently with conventional therapy.

In certain embodiments, the invention provides a method for improving the safety and efficacy of a treatment with lenalidomide (Revlimid®), wherein the method comprises administering a CDK4/CDK6 inhibitor to the patient being treated with lenalidomide (Revlimid®). In certain embodiments, safety is improved if any side effects of treatment with lenalidomide is reduced by at least 10%, 20%, 30%, 40%, 50%, 60%, 70%, 80%, 90%, 100%, 150%, 200%, 250%, 300%, 350%, 400%, 450%, 500%, 600%, 700%, 800%, 900%, or by at least 1000%.

In certain embodiments, Revlimid® and a second active agent (e.g., a CDK4/CDK6 inhibitor) are cyclically administered to a patient. Cycling therapy involves the administration of an active agent for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment.

Consequently, in one specific embodiment of the invention, lenalidomide (Revlimid®) and a second active agent is administered daily in a single or divided doses in a four to six week cycle with a rest period of about a week or two weeks. The invention further allows the frequency, number, and length of dosing cycles to be increased. Thus, another specific embodiment of the invention encompasses the administration of Revlimid® and a second active agent (e.g., a CDK4/CDK6 inhibitor) for more cycles than are typical when it is administered alone. In yet another specific embodiment of the invention, Revlimid® and a second active agent (e.g., a CDK4/CDK6 inhibitor) are administered for a greater number of cycles that would typically cause dose-limiting toxicity in a patient to whom a second active ingredient is not also being administered.

In one embodiment, Revlimid® is administered daily and continuously for three or four weeks at a dose of from about 5 to about 25 mg/d followed by a break of one or two weeks, in combination with a CDK4/CDK6 inhibitor. In a particular embodiment, Revlimid® is administered (in combination with a CDK4/CDK6 inhibitor) in an amount of about 5, 10, or 25 mg/day, preferably in an amount of about 25 mg/day for three to four weeks, followed by one week or two weeks of rest in a four or six week cycle.

In one embodiment of the invention, a CDK4/CDK6 inhibitor is administered orally, with administration of Revlimid® during a cycle of four to six weeks. In a specific embodiment, one cycle comprises the administration of from about 5 to about 25 mg per day of Revlimid® for three to four weeks and then one or two weeks of rest, and the administration of from about 5 to about 200 mg per day of a CDK4/CDK6 inhibitor for three to four weeks and then one or two weeks of rest. In a specific embodiment, PD-0332991 may be administered daily for 21 days in 28-day cycles to patients in successive dose escalating cohorts at doses from 25 mg to 150 mg daily. An alternative schedule of 14 days dosing in 21-day cycles comprises administration of PD-0332991 in an amount of from about 100 mg to about 225 mg daily.

In certain embodiments, a method of treatment described herein comprises administering to a patient Revlimid®, a CDK4/CDK6 inhibitor and one or more additional active agents, wherein the one or more active agents include, without limitation, melphalan, prednisone, dexamethasone, vincristine, doxorubicin, Velcade® (bortezomib), Dacogen® (decitabine), and bisphosphonate. In one embodiment, the CDK4/CDK6 is administered with one additional active agent. In another embodiment, the CDK4/CDK6 is administered with two additional active agents. In another embodiment, the CDK4/CDK6 is administered with three additional active agents. In another embodiment, the CDK4/CDK6 is administered with more than three additional active agents.

In a specific embodiment, a method of treatment described herein comprises administering to a patient Revlimid® (lenalidomide), a CDK4/CDK6 inhibitor and dexamethasone. In a specific embodiment, a method of treatment described herein comprises administering to a patient Revlimid® (lenalidomide, 5 to about 25 mg per day on Days 1-21 in a 28 days cycle), a CDK4/CDK6 inhibitor (25 to about 150 mg per day on Days 1-21 in a 28 days cycle), and a low dose regimen of dexamethasone (40 mg/day on Days 1, 8, 15 and 22 in a 28 days cycle). In another specific embodiment, a method of treatment described herein comprises administering to a patient Revlimid® (lenalidomide, 5 to about 25 mg per day on Days 1-21 in a 28 days cycle), a CDK4/CDK6 inhibitor (25 to about 150 mg per day on Days 1-21 in a 28 days cycle), and a high dose regimen of dexamethasone (40 mg/day on Days 1-4, 9-12, 17-20 in a 28 days cycle).

In another specific embodiment, a method of treatment described herein comprises administering to a patient a CDK4/CDK6 inhibitor, Revlimid® (lenalidomide) and melphalan. In another specific embodiment, a method of treatment described herein comprises administering to a patient a CDK4/CDK6 inhibitor, Revlimid® (lenalidomide), and prednisone. In another specific embodiment, a method of treatment described herein comprises administering to a patient a CDK4/CDK6 inhibitor, Revlimid® (lenalidomide), melphalan and prednisone.

In a specific embodiment, a method of treatment described herein comprises administering to a patient Revlimid® (lenalidomide), a CDK4/CDK6 inhibitor, and Velcade® (bortezomib). In another specific embodiment, a method of treatment described herein comprises administering to a patient a CDK4/CDK6 inhibitor, Revlimid® (lenalidomide), and cyclophosphamide.

In accordance with the methods of treatment described herein which comprise administering to a patient Revlimid® (lenalidomide), a CDK4/CDK6 inhibitor and one or more additional active agents, any dose of the active agents deemed suitable for administration or known in the art to treat multiple myeloma can be used.

In a specific embodiment, a method of treatment described herein comprises administering to a patient Revlimid® (lenalidomide), a CDK4/CDK6 inhibitor and cyclophosphamide. In another specific embodiment, a method of treatment described herein comprises administering to a patient a CDK4/CDK6 inhibitor, Revlimid® (lenalidomide), and bisphosphonate.

Exemplary doses ranges of Revlimid® (lenalidomide) include, but are not limited to, 25 mg, 5 to 10 mg, 5 to 25 mg, or 5 to 50 mg. In certain embodiments, Revlimid® (lenalidomide) may be administered at a dose of 5 to 25 mg per day.

Exemplary doses of melphalan include, but are not limited to, 0.01 mg/kg, 0.05 mg/kg, 0.1 mg/kg, 0.15 mg/kg, 0.16 mg/kg, 0.17 mg/kg, 0.18 mg/kg, 0.19 mg/kg, 0.2 mg/kg, 0.25 mg/kg, 0.3 mg/kg, 0.35 mg/kg, 0.4 mg/kg, 0.45 mg/kg, or 0.5 mg/kg. Exemplary dose ranges of melphalan include, but are not limited to, 0.01 to 0.05 mg/kg, 0.05 to 0.1 mg/kg, 0.1 to 0.15 mg/kg, 0.1 to 0.2 mg/kg, 0.1 to 0.25 mg/kg, 0.2 to 0.3 mg/kg, 0.2 to 0.35 mg/kg, 0.3 to 0.4 mg/kg, 0.3 to 0.45 mg/kg, or 0.4 to 0.5 mg/kg. In certain embodiments, melphalan may be administered at a dose of 0.1 to 0.25 mg/kg per day.

Exemplary doses of prednisone include, but are not limited to, 0.1 mg/kg, 0.5 mg/kg, 1 mg/kg, 1.5 mg/kg, 2 mg/kg, 2.5 mg/kg, 3 mg/kg, 3.5 mg/kg, 4 mg/kg, 4.5 mg/kg, or 5 mg/kg. Exemplary dose ranges of prednisone include, but are not limited to, 0.1 to 1 mg/kg, 0.1 to 2 mg/kg, 1 to 2 mg/kg, 1 to 3 mg/kg, 2 to 3 mg/kg, 2 to 4 mg/kg, 2 to 5, or 3 to 5 mg/kg. In certain embodiments, prednisone may be administered at a dose of 1 to 3 mg/kg per day.

Exemplary doses of dexamethasone include, but are not limited to, 5 mg, 10 mg, 15 mg, 20 mg, 25 mg, 30 mg, 35 mg, 40 mg, 45 mg, 50 mg, 55 mg, 60 mg, 65 mg, or 70 mg. Exemplary dose ranges of dexamethasone include, but are not limited to, 5 to 20 mg, 10 to 30 mg, 20 to 40 mg, 30 to 50 mg, 40 to 60 mg, or 50 to 70 mg. In certain embodiments, dexamethasone may be administered at a dose of 40 mg on days 1 to 4, 9-12, and 17-20 of a 28-days treatment cycle (i.e., high-dose dexamethasone treatment). In other embodiments, dexamethasone may be administered at a dose of 40 mg on days 1, 8, 15, and 22 of a 28-days treatment cycle (i.e., low-dose dexamethasone treatment).

Exemplary doses of Velcade® (bortezomib) include, but are not limited to, 0.1 mg/m² (of body surface area), 0.5 mg/m², 0.6 mg/m², 0.7 mg/m², 0.8 mg/m², 0.9 mg/m², 1 mg/m², 1.1 mg/m², 1.2 mg/m², 1.3 mg/m², 1.4 mg/m², 1.5 mg/m², 2 mg/m², 2.5 mg/m², or 3 mg/m². Exemplary dose ranges of Velcade® (bortezomib) include, but are not limited to, 0.1 to 0.5 mg/m², 0.5 to 1 mg/m², 1 to 1.5 mg/m², 1 to 2 mg/m², 1.5 to 2.5 mg/m², or 2 to 3 mg/m². In certain embodiments, Velcade® (bortezomib) may be administered at a dose of 0.7 to 1.3 mg/m² daily, every other day, every third day, or weekly.

Exemplary doses of Dacogen® (decitabine) include, but are not limited to, 5 mg/m² (of body surface area), 10 mg/m², 15 mg/m², 20 mg/m², or 25 mg/m². Exemplary dose ranges of Dacogen® (decitabine) include, but are not limited to, 5 to 10 mg/m², 5 to 15 mg/m², 10 to 15 mg/m², 10 to 20 mg/m², 15 to 20 mg/m², or 15 to 25 mg/m². In certain embodiments, Dacogen® (decitabine) may be administered at a dose of 10 to 20 mg/m² daily for five days.

Exemplary doses of bisphosphonate include, but are not limited to 1 mg, 5 mg, 10 mg, 15 mg, 20 mg, 25 mg, 30 mg, 40 mg, 50 mg, 60 mg, 70 mg, 80 mg, 90 mg, 100 mg, 110 mg, 120 mg, 130 mg, 140 mg, 150 mg, or 200 mg. Exemplary dose ranges of bisphosphonate include, but are not limited to, 1 to 10 mg, 1 to 25 mg, 1 to 50 mg, 10 to 25 mg, 10 to 50 mg, 25 to 50 mg, 25 to 75 mg, 50 to 75 mg, 50 to 100 mg, 75 to 100 mg, 75 to 150 mg, 100 to 150 mg, 100 to 200 mg, or 150 to 200 mg.

In certain embodiments, the CDK4/CDK6 inhibitor is dosed at amounts of from about 5 to about 150 mg daily for 21 days in 28-day cycles. In certain embodiments, the CDK4/CDK6 inhibitor is dosed at amounts of 5 mg, 10 mg, 25 mg, 50 mg, 75 mg, 100 mg or 125 mg daily.

6.5 Pharmaceutical Compositions

In certain embodiments, immunomodulatory compounds (e.g., Revlimid®, lenalidomide) and the second active agent are formulated separately. In other embodiments, immunomodulatory compounds (e.g., Revlimid®, lenalidomide) and the second active agent are formulated together.

In certain embodiments, a therapeutic method of the invention includes administering Revlimid® (lenalidomide) and the second active agent systemically, or locally as an implant or device. When administered, the therapeutic composition for use in this invention is in a pyrogen-free, physiologically acceptable form.

In certain embodiments, the invention provides a pharmaceutical composition comprising Revlimid® (lenalidomide) and the second active agent. In certain embodiments, the invention provides a pharmaceutical composition comprising Revlimid® (lenalidomide), the second active agent, and a pharmaceutically acceptable carrier, diluent or excipient.

In solid dosage forms for oral administration (capsules, tablets, pills, dragees, powders, granules, and the like), one or more therapeutic compounds may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

The compositions for use with the methods the invention may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Pharmaceutical compositions and dosage forms to be used with the methods of the invention comprise immunomodulatory compound, or a pharmaceutically acceptable salt, solvate, hydrate, stereoisomer, clathrate, or prodrug thereof. Pharmaceutical compositions and dosage forms can further comprise one or more excipients.

Single unit dosage forms are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape, and type of dosage forms will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, pharmaceutical compositions and dosage forms can contain little, if any, lactose other mono- or di-saccharides. As used herein, the term “lactose-free” means that the amount of lactose present, if any, is insufficient to substantially increase the degradation rate of an active ingredient.

Lactose-free compositions comprise excipients that are well known in the art and are listed, for example, in the U.S. Pharmacopeia (USP) 25 NF20 (2002). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

Anhydrous pharmaceutical compositions and dosage forms comprising a second active agent can also be used, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Like the amounts and types of excipients, the amounts and specific types of active ingredients in a dosage form may differ depending on factors such as, but not limited to, the route by which it is to be administered to patients. However, typical dosage forms comprise an immunomodulatory compound or a pharmaceutically acceptable salt, solvate, hydrate, stereoisomer, clathrate, or prodrug thereof in an amount of from about 0.10 to about 150 mg. Typical dosage forms comprise an immunomodulatory compound or a pharmaceutically acceptable salt, solvate, hydrate, stereoisomer, clathrate, or prodrug thereof in an amount of about 0.1, 1, 2, 5, 7.5, 10, 12.5, 15, 17.5, 20, 25, 50, 100, 150 or 200 mg. In a particular embodiment, a preferred dosage form comprises 4-(amino)-2-(2,6-dioxo(3-piperidyl))-isoindoline-1,3-dione in an amount of about 0.1, 0.2, 0.5, 1, 2, 2.5, 3 or 5 mg. In a specific embodiment, a preferred dosage form comprises lenalidomide in an amount of about 5, 10, 15, 20, 25 or 50 mg. Typical dosage forms comprise the second active agent in an amount of 1 to about 1000 mg, from about 5 to about 500 mg, from about 10 to about 350 mg, or from about 50 to about 200 mg. Of course, the specific amount of the anti-cancer drug will depend on the specific agent used, the type of cancer being treated or managed, and the amount(s) of an immunomodulatory compound and any optional additional active agents concurrently administered to the patient.

Pharmaceutical compositions that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions is typically present in from about 50 to about 99 weight percent of the pharmaceutical composition or dosage form.

Disintegrants are used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

A preferred solid oral dosage form comprises an immunomodulatory compound, anhydrous lactose, microcrystalline cellulose, polyvinylpyrrolidone, stearic acid, colloidal anhydrous silica, and gelatin.

A second active agent can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the second active agent. Single unit dosage forms suitable for oral administration such as, but not limited to, tablets, capsules, gelcaps, and caplets that are adapted for controlled-release can be used with the methods and compositions of the present invention.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of a second active agent can also be incorporated into the parenteral dosage forms. For example, cyclodextrin and its derivatives can be used to increase the solubility of an immunomodulatory compound and its derivatives. See, e.g., U.S. Pat. No. 5,134,127, which is incorporated herein by reference.

Topical and mucosal dosage forms include, but are not limited to, sprays, aerosols, solutions, emulsions, suspensions, eye drops or other ophthalmic preparations, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms that can be used are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980 & 1990).

The pH of a pharmaceutical composition or dosage form may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

6.6 Kits

In certain embodiments, lenalidomide and a second active agent are not administered to a patient at the same time or by the same route of administration. This invention therefore encompasses kits which, when used by the medical practitioner, can simplify the administration of appropriate amounts of active ingredients to a patient.

A typical kit of the invention comprises a dosage form of Revlimid® and a dosage form of a second active agent, or a pharmaceutically acceptable salt, solvate, hydrate, stereoisomer, prodrug, or clathrate thereof. In a specific embodiment, a kit of the invention comprises a dosage form of a CDK4/CDK6 inhibitor and a dosage form of Revlimid®.

Kits of the invention can further comprise devices that are used to administer the active ingredients. Examples of such devices include, but are not limited to, syringes, drip bags, patches, and inhalers.

### EXAMPLES

### 7.1 Example 1

### Combination Therapy of Lenalidomide and Dexamethasone

Lenalidomide (Revlimid®) has a dual mechanism of action; it exerts a direct cytotoxic effect on myeloma cells and modifies the tumor microenvironment. Chanan-Khan A, et al. J. Clin. Oncol. 2008; 26:1544-1552. Lenalidomide (Revlimid®) induces tumor suppressor genes, arrests cell cycle resulting in cancer cell apoptosis, and activates caspases in multiple myeloma. Lenalidomide (Revlimid®) activates CD4+, CD8+ T cells, NK and NKT cells in multiple myeloma. Lenalidomide (Revlimid®) augments humoral immunity in multiple myeloma.

Lenalidomide is currently indicated in combination with dexamethasone for the treatment of multiple myeloma patients who have received at least one prior therapy. Lenalidomide [package insert]. Summit, N.J.: Celgene Corp. 2009.

In newly diagnosed multiple myeloma, most of patients responded to lenalidomide (Revlimid®) (25 mg/day, days 1-21 in a 28 days cycle) in combination with dexamethasone (40 mg/day, days 1-4, 9-12, 17-20 in a 28 days cycle; or 40 mg/day, days 1, 8, 15 and 22 in a 28 days cycle). The combination therapy yielded high overall response rate and complete response; significantly increased progression-free survival at 1 year; improved 1-year progression-free survival for patients with high-risk cytogenetic factors; and effective salvage was achieved through crossover. The combination therapy was well tolerated. Most adverse events were manageable with dose modification. The combination therapy is an effective first-line therapy for the treatment of patients with newly diagnosed multiple myeloma.

### 7.2 Example 2

### Combination Therapy of Lenalidomide and CDK4/6 Inhibitor

Lenalidomide and a CDK4/CDK6 inhibitor control tumor expansion and enhance tumor killing in the treatment of multiple myeloma. Patients with multiple myeloma are treated with lenalidomide (Revlimid®, 5 to 25 mg/day orally on days 1-21 in a 28 days cycle) and a CDK4/CDK6 inhibitor (PD 0332991, 25 to 150 mg/day orally on days 1-21 in a 28 days cycle). Maintenance treatment are continued until the disease progression. The therapy using Revlimid® in combination with a CDK4/CDK6 inhibitor is highly active and well tolerated in multiple myeloma patients whose prognosis is otherwise poor.

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

1. A method for treating or preventing multiple myeloma in a human, wherein the method comprises administering to a human in need thereof cyclin-dependent kinase (CDK) inhibitor and administering 3-(4-amino-1-oxo-1,3-dihydro-isoindol-2-yl)-piperidine-2,6-dione.

2. The method of claim 1, wherein the cyclin-dependent kinase inhibitor is a cyclin-dependent kinase 4 (CDK4) inhibitor, cyclin-dependent kinase 6 (CDK6) inhibitor, or cyclin-dependent kinase 4 and 6 (CDK4/6) inhibitor.

3. The method of claim 2, the cyclin-dependent kinase inhibitor is CDK4/6 inhibitor.

4. The method of claim 1, 2, or 3, wherein the CDK inhibitor is administered orally.

5. The method of claim 1, 2, or 3, wherein 3-(4-amino-1-oxo-1,3-dihydro-isoindol-2-yl)-piperidine-2,6-dione is administered orally.

6. The method of claim 5, wherein 3-(4-amino-1-oxo-1,3-dihydro-isoindol-2-yl)-piperidine-2,6-dione is administered in the form of a capsule or tablet.

7. The method of claim 5, wherein 3-(4-amino-1-oxo-1,3-dihydro-isoindol-2-yl)-piperidine-2,6-dione is administered in an amount of from about 10 to about 25 mg per day.

8. The method of claim 1, wherein the CDK inhibitor and 3-(4-amino-1-oxo-1,3-dihydro-isoindol-2-yl)-piperidine-2,6-dione are administered at the same time.

9. The method of claim 1, wherein the CDK inhibitor and 3-(4-amino-1-oxo-1,3-dihydro-isoindol-2-yl)-piperidine-2,6-dione are administered sequentially.

10. A method for improving the safety of a treatment with 3-(4-amino-1-oxo-1,3-dihydro-isoindol-2-yl)-piperidine-2,6-dione), wherein the method comprises administering a CDK inhibitor to a patient being treated with 3-(4-amino-1-oxo-1,3-dihydro-isoindol-2-yl)-piperidine-2,6-dione.
